(19) United States
(12) Patent Application Publication
(10) Pub. No.: US 2008/0081980 A1
(43) Pub. Date: Apr. 3, 2008

(54) Apparatus and process for stroke examination and treatment using a C-arch X-ray system

(76) Inventors: Michael Maschke, Lonnerstadt (DE); Gunter Lauritsch, Erlangen (DE)

Correspondence Address: BRINKS HOFER GILSON & LIONE, P.O. BOX 10395, CHICAGO, IL, 60610, US

(21) Appl. No.: 11/522,605

(22) Filed: Sep. 18, 2006

(51) Int. Cl. A61B 5/00

(52) U.S. Cl. 600/407; 600/301

(57) ABSTRACT

A medical treatment suite is described, having one or more imaging modalities, such as a X-ray, computer tomography (CT) magnetic resonance imaging (MRI), or the like, with additional sensors such as ultrasound imaging, a patient motion sensor, a body parameters monitor, a blood chemistry monitor and imaging processing modules or functions such that a composite image from the image sensors and other patient information from the sensors may be displayed for the purpose of identifying a type of stroke in a patient. After determining the type of treatment protocol to be used, the treatment suite may be used to monitor the administration of the treatment and the patient condition. The sensors communicate with the treatment suite by data interface, and the data obtained by the treatment suite may be transmitted to a remote location for viewing or storage.

### TECHNICAL FIELD

The present application relates of an apparatus and method for assisting in the diagnosis of the type of stroke suffered by a patient.

### BACKGROUND

Stroke is one of the most common and significant vascular disorders. Worldwide, the syndrome known as stroke is in second place amongst the causes of death. For both the patients and their relatives, a stroke means wide-ranging burdens. As of a year after becoming ill, only about 40% of stroke survivors are without restrictions in their daily activities. Only half of the patients in whom the neurological problems typical of a stroke have occurred reach the emergency room within the therapeutic window of 3 hours.

A stroke can be due to risk factors, some of which can be influenced and others which cannot. The risk factors for vascular disorders (stroke, heart attack, arteriosclerosis) have a mutual influence on one another, and this adverse interplay increases the overall risk. Health care expenses can be controlled by preventive reduction of the risk factors that can be changed and by rapid treatment, for instance in an ischemic stroke by thrombolytic therapy using rTPA (recombinant tissue plasminogen activator) within 3 hours after the stroke.

When a patient is received by a hospital, the medical staff takes the data and anamnesis (complete history of the disease as the patient himself describes it), in order to determine the next steps in diagnosis and treatment. With this assessment, the suspicion can be confirmed that the symptoms can be ascribed to a stroke and not to a systematic disease, such as low blood sugar or some other neurological disorder.

In addition, in the emergency room an initial diagnosis is made, which includes an EKG to exclude relevant cardiac irregularities, a sonogram of the carotid arteries to detect severe stenoses or occlusions, and various laboratory tests. Thus, symptoms similar to those in a stroke can also be ascribed to altered blood sugar and electrolyte values. Even metabolic changes in liver or kidney failure can produce similar symptoms. In addition, laboratory tests provide information about the condition of the corpuscles and the blood coagulation system.

In a stroke, a distinction is made between the ischemic form (cerebral infarction) and the hemorrhagic form (cerebral hemorrhage). In both kinds, the supply of blood to the brain is hindered, which causes nerve cells to die. Ischemic stroke is caused by an occlusion, that is, a blockage of a cerebral artery. The artery becomes clogged either by a thrombus, that is, a blood clot, or by an embolus, that is, a small clump that has migrated from some other place in the body. Approximately three-fourths of all stroke patients suffer an ischemic stroke. A hemorrhagic stroke is caused by intracerebral bleeding, in which blood from a blood vessel escapes into the surrounding brain tissue. Besides the resultant interruption in blood supply, which causes the death of nerve cells, the accumulating blood also increases the pressure on the brain tissue, which further speeds up nerve cell death. Approximately one-fourth of stroke patients suffer a hemorrhagic stroke.

The forms of treatment for the two types of stroke differ considerably. In ischemic stroke, circulation must be promoted, while in hemorrhagic stroke bleeding must be stopped. For an ischemic stroke, this is most effectively done by thrombolytic therapy using rTPA (recombinant tissue plasminogen activator). However, this type of therapy would be contraindicated in a hemorrhagic stroke.

In a hemorrhagic stroke, the blood can be removed from the brain by centesis to lower the pressure inside the skull. In the case of bleeding from a burst aneurysm, surgical intervention may be needed. Treatment for hemorrhagic stroke requires not only the implantation of probes to measure the cerebral pressure but also pressure-relieving trepanation or shunt implantation. Occasionally, the bleeding can be lessened or stopped with medications that promote blood coagulation. In the case of subarachnoid bleeding or bleeding from burst cerebral aneurysms, not only conservative treatment options but neurosurgical interventions as either early or delayed operations are used, which are intended to close the source of bleeding from the ruptured aneurysm by the placement of a metal clip.

In the treatment courses and guidelines that are currently employed, the history and physical examination are followed by a CT scan, in order to detect ischemic stroke and to exclude hemorrhagic stroke.

The known treatment paths have a disadvantage that, in the patient with hemorrhagic stroke, a great deal of time is lost when obtaining the CT scan, and after that the patient must still be transported to a surgical or neurological intervention room in order to stop the bleeding. During the CT, intervention is difficult, because of the poor accessibility to the patient.

Methods and apparatuses for angiographic and soft tissue 3D images with the aid of a C-arch X-ray system are known. For instance, 3D images of the skull and the vessels can be made with a Siemens AXIOM ARTIS FA/FB, where contrast agents are injected into the vessels.

### BRIEF DESCRIPTION OF THE DRAWING

FIG. 1 is a block diagram of an example of the sensors, signal and data processing and interfaces of a treatment suite of an embodiment.

### DETAILED DESCRIPTION

Exemplary embodiments may be better understood with reference to the drawing. In the interest of clarity, not all the routine features of the implementations described herein are described. It will of course be appreciated that in the development of any such actual implementation, numerous implementation-specific decisions must be made to achieve a developers' specific goals, such as compliance with system and business related constraints, and that these goals will vary from one implementation to another.

A “stroke therapy unit”, in which the patient need not be transported from place-to-place between the individual steps in diagnosis and therapy is described. Such a stroke therapy unit may include the following equipment types integrated as a platform for performing diagnosis and monitoring of a patient:
an imaging modality; and
one or more of:
   an image processor for at least one of soft tissue or angiographic data obtained by the imaging modality;
   a sensor for detecting patient motion;
   a processor for motion correction of images for patient motions;
   a patient monitor;
   an image fusion unit;
   a blood sugar analysis device;
   a blood analysis device;
   a computer and interface for entering patient data; and
   a data interface with a local area network or a wide area network.

The imaging modality may be a C-arch X-ray unit or other imaging modalities, such as CT (Computerized Tomography), MRI (Magnetic Resonance Imaging), PET scan (Positron Emission Tomography), SPECT (Single Photon Emission Computer Tomography), an ultrasound device, or the like, or later developed imaging technologies.

The combination of hardware and software to accomplish the tasks described herein may be termed a platform or “therapy unit”. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figure or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, including both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a corresponding receiving device. While the communication is described as being from a transmitter to a receiver, this does not exclude the reverse path, and a wireless communications device may include both transmitting and receiving functions.

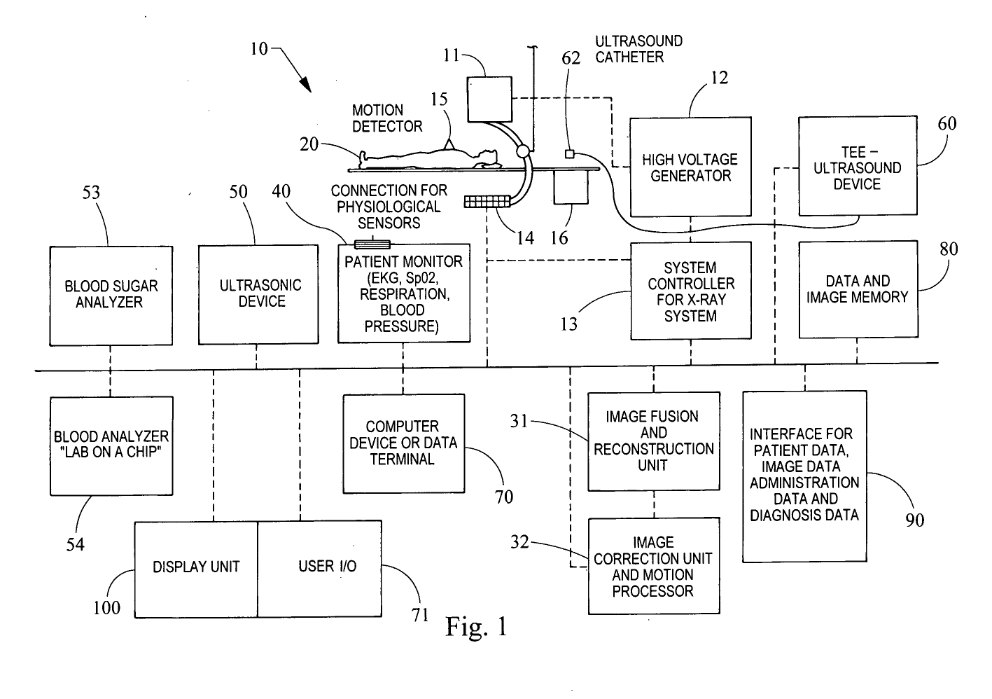

FIG. 1 shows a block diagram of an example of a therapy unit. Other embodiments of the therapy unit may include fewer than all of the devices, or functions, shown in FIG. 1. A C-arch X-ray device 10 is representative of the imaging modalities which may be used. The C-arch X-ray device 10 is rotated such that a sequence of projection X-ray images is obtained by an X-ray detector 14 positioned on an opposite side of the patient 20 from the X-ray source 11, and the images are reconstructed by any technique of processing for realizing tomographic images. Additional, different, or fewer components may be provided. The devices and functions shown are representative, but not inclusive. The individual units, devices, or functions may communicate with each other over cables or in a wireless manner, and the use of dashed lines for some of the connections is intended to suggest that alternative means of connectivity may be used.

The C-arch or C-arm X-ray radiographic unit and the associated image processing may be of the type described in US PG-Pub Application US2006/0120507, entitled “Angiographic X-ray Diagnostic Device for Rotational Angiography, filed on Nov. 21, 2005, which is incorporated herein by reference. Such an apparatus may produce angiographic and soft tissue tomographic images comparable to, for example, CT equipment, while permitting more convenient access to the patient for treatment procedures.

The sensor portions of the therapy unit may be located in a therapy room, and some or all of the signal and data processing and data display may also be located in the therapy room; however, some or all of the equipment and functionality not directly associated with the sensing of the patient, may be remotely located. Such remote location is facilitated by high speed data communications on local area networks, wide area networks, and the Internet. The signals representing the data and images may be transmitted by modulation of representations of the data on electromagnetic signals such as light waves, radio waves, or signals propagating on wired connections.

The therapy unit may thus be located remotely from the specialists making the diagnosis and for determining the appropriate course of treatment. Of course, the specialists may be present with the patient as well.

FIG. 1 illustrates some of the equipment and functions which may be provided. The X-ray apparatus 10 may include a radiation source 11, with an associated high-voltage generator 12, a control system 13 and a detector 14. When an X-ray system such as the AXIOM Artis dTA DynaCT (available from Siemens AG, Erlangen, Germany) is used to perform angiographic computerized tomography (ACT), computer tomography (CT)-like images may be obtained during a procedure. In such a use, image acquisition may take approximately 10 seconds with C-arm rotation through approximately 200 degrees.

A patient support table 16 may be used for some or all of the examination steps and thus may transfer the patient 20 between various sensors or otherwise position the patient 20. The imaging apparatus may be a C-arch apparatus, with robotic positioning, or any suitable X-ray device or other electromagnetic imaging modality such as a CT, MRI and PET scanner or an ultrasound device, and more than one imaging modality may be used for diagnostic imaging and treatment purposes. An X-ray device, for example, may also be used during the treatment phase.

A motion sensor 15 may be provided for detecting motion of the patient 20 during an exam and taking the motion into account in a motion processor 32 prior to the image reconstruction processing 31. The motion sensor 15 may be a mathematical motion detector, for instance deriving from the image signals themselves, such as is described as in US Patent Application 2002/0163994, “In-Line Correction of Patient Motion in Three-Dimensional Positron Emission Tomography”. In another aspect, the motion sensor may be capacitive as in U.S. Pat. No. 6,661,240, “Method and System for Capacitive Motion Sensing and Position Control”; magnetic, as in EP 0993804, “Method and system for tracking an object; acoustical, as in EP 1034738; “Positioning based on ultrasound emission”; or, optical, where the position of the patient may be detected by an optical or infrared camera and by computational methods of pattern recognition. The patient can be scanned with a laser beam. Patient displacements or shifts are ascertained and corrected in an image processing unit 32. Processing units may be combined into a single processor, or be multiple processors, and a processing function may be represented as either hardware, software, or a combination thereof.

The motion sensor 15 may transmit data to the image processing unit 32 with through a wired connection or in wireless form. Before the beginning of the exam, the motion sensor 15 may be calibrated relative to the spatial coordinates of the various examination apparatus and may be calibrated relative to the patient support table 16.

The function of eliminating motion artifacts may include motions that are due to breathing and the motion of the heart (for example, by “ECG gating”) and the blood vessels. For eliminating the breathing artifacts, a chest belt may be used by employing suitable sensors to ascertain the breathing amplitude and frequency and initiate corrective calculations in the image processing unit 32 that minimize the motion artifacts in the reconstructed image. Alternatively, the amplitude and frequency of breathing may be calculated from an envelope curve of the electrocardiogram (EKG) signal and provided to the image processor 32 or the image fusion unit 31.

A patient monitor 40, such as described in U.S. Pat. No. 6,221,012, “Transportable Modular Patient Monitor with Data Acquisition Modules”; or as a product, the Infinity Gamma (available from Drätger Medical Deutschland GmBH, Lübeck, Germany), may sense the blood pressure, heart rate, oxygen saturation, and EKG and the data may be stored in a memory 80, along with image and other data obtained from the various sensors.

An ultrasound device 50, for instance, the iLook device from Sonosite, http://www.sonosite.com/ and/or the HandyScan from Primedic, may be employed to perform duplex sonography of the cardiac arteries for detecting severe stenoses or occlusions, a preliminary diagnosis of brain bleeds and the setting and guidance of a centesis nozzle. In addition, with the ultrasound device 50, the progress of thrombolytic therapy can be followed, without emitting X-radiation.

The patient monitor 40 and the ultrasound device 50, for example, may be combined into one unit, as described for instance in US Patent Application 2004/0249279, “Patient Monitor for Processing Signals from an Ultrasound Probe”. In another aspect, the patient monitor 40, the ultrasound device 50 and a defibrillator (not shown) may be combined into a unit, as described in German patent application 2005P04026, not yet published, Serial No. 102005031642.5.

A stroke may be triggered by atrial fibrillation. Emboli can originate in the venous system, as in the case of deep vein thrombosis. A cerebral vascular embolus may also occur in patients with cardiac shunts, atrial septum defects, or a persistent foramen ovale. Therefore, a TEE exam could be performed, especially for patients with atrial fibrillation. Thus, a suitable TEE ultrasound apparatus 60 for performing transesophageal echocardiography may be integrated with the remainder of the equipment. A TEE apparatus is known, for example, from U.S. Pat. No. 6,142,941, “Device for Carrying Out a Transoesophageal Echocardiography and a Cardioversion.”

In another aspect, an ACUNAV catheter 62 from Siemens AG (Erlangen, Germany) can be used. This catheter is advanced through the venous system into the heart and can be used for generating ultrasound images from the chambers of the heart. An ultrasound device to which both sound heads or catheters for TEE examination and extracorporeal sound heads may be provided.

An image fusion unit 31 or function (recording, segmenting, superimposing) may combine information from different imaging devices. For example, sonograms can be fused with the x-ray and angiographic images in 2D, 3D or 4D image representations.

A compact blood sugar analysis device 53, such as Accu-Check from Roche Diagnostics GmbH (Mannheim, Germany) may be used for determining the blood sugar values. In addition, a blood analysis device 54, such as “Lab on a Chip”, which is being developed by Siemens AG, may be used for determining further blood values or certain genetic or molecular markers (see, for example, WO 00/56922, “Genetic Polymorphism and Polymorphic Pattern for Assessing Disease Status, and Compositions for Use Thereof”, and DE 69919885, “Method for Measuring Cellular Adhesion” for gene tests and tests with molecular markers for stroke). See also, WO 2005/106024, entitled “method and Assembly for DNA Isolation with Dry Reagents” and WO 2005/106023, entitled “PCR Process and Arrangement for DNA Amplification using Dry Reagents”, as examples of devices and methods which may be used. As medical knowledge increases, further test devices and methods may be added to the treatment suite.

A computer device 70 may be a notebook, such as a SIMpad (Siemens AG, Erlangen, Germany) or other processing device with which the demographic, history, diagnosis and/or therapy data of the patient can be recorded, called up and sent to and from the medical information management system of the hospital. The computer device 70 may be provided with an interface for reading out the data from an HMO (health maintenance organization) or health insurance or card, and may be connected to the remainder of the treatment suite by a wireless connection. A user input device 71, such as a keyboard, computer display device, and mouse, may be provided for manual input and control. In addition, the examination and therapy actions already performed may be documented in this computer device, including the medications administered or still to be administered. Some or all of the data may be forwarded to another entity for use in diagnosis, billing and administrative purposes, or further image processing and storage using known interfaces such as DICOM and SOARIAN, or special purpose or later developed data formatting and processing techniques. SOARIAN is a web-browser-based information management system for medical use, integrating clinical, financial, image, and patient management functions and facilitating retrieval and storage of patient information and the performance of analytic tasks (available from Siemens Medical Solutions Health Service Corporation, Malvern, Pa.).

The stroke therapy unit may be operated by the acts summarized below in any desired order. Different, additional, or fewer acts may be performed

The patient may be brought to the stroke therapy unit, and identified. Data for patient identification is obtained either manually or by reading a health insurance or other identification card via an interface, such as DICOM (Digital Communications in Medicine) or, optionally, interviewing the patient and inputting demographic and medical history data, or retrieving such data if previously stored in an accessible memory. The patient is automatically or manually assigned a patient an identification number (ID) if such a number is not already associated with the patient. Sensors from the patient monitor are attached, and patient parameters, such as EKG, blood pressure and SpO₂, are measured and recorded. Blood sugar values are measured and recorded, or existing laboratory test results are recalled. Further blood values (such as coagulation factors) and markers may be measured or recalled. The patient is positioned on the examination table. An ultrasound examination, optionally including using an ultrasonic contrast agent, is performed and recorded. A CT (computerized tomography), X-ray or other imaging exam is performed and recorded. Rotational X-ray images over at least an azimuth of 180° using at least two projection images, with or without a contrast agent, may be obtained and recorded using a C-arch X-ray apparatus. The image data obtained may be corrected for motion artifacts. 3D volumetric images or 2D images may be generated or reconstructed. The images may be displayed on a monitor or by a projector.

Optionally, the soft tissue and contrast-agent-reinforced images may be fused by, for example segmenting, recording, or superimposing in the image processor 31. The resultant data and images (2D, 3D, 4D, superimposed, segmented, or the like) from the various sensors individually or merged may be displayed on a visual display 100, which may be located in the treatment suite, or replicated in a similar manner at a remote location, which may be at any physical distance from the patient.

In another embodiment, a method of diagnosing a stroke includes the acts of providing a processor configured to receive and process information relating to patient identification, to measure and record parameters obtained from sensors attached to the patient, and to measure or recall blood sugar values. Additional data may be received from an ultrasound device or an imaging modality using electromagnetic radiation (including X-rays). The imaging data may be corrected using data from a motion sensor and formed into, for example, a 3D volumetric image of at least a portion of the patient. The image may be transmitted to a display monitor or projector.

In an aspect, the ultrasound imaging portion of the method may be employed to, for example, exclude brain bleeds, and to perform a cartoid exam for identifying stenoses. Alternatively, a radiological exam may be used for similar purposes. Transesophegal echocardiography (TEE) may also be performed. The CT or C-arm X-ray CT exam may be performed without a contrast agent for finding a bleed (treatment path A) or excluding a bleed (treatment path B). Alternatively, or supplementally, a neurological radiological exam of the skull using a contrast agent may be performed for finding a bleed (treatment path A), or for excluding a bleed (treatment path B).

Treatment path A may be used for a hemorrhagic stroke and may include removing the blood from the brain by centesis to lower the pressure inside the skull. In the case of bleeding from a burst aneurysm, the affected vessel may also be operated on. Surgical intervention may include implantation of probes to measure the cerebral pressure (connectable, for example to the patient monitor) and pressure-relieving trepanation. In neurosurgery, trepanation involves the surgical opening of the skull, either to perform surgical interventions in the interior of the skull or to lower the internal pressure of the skull. Optionally, the bleeding is reduced or stopped with medications that promote blood coagulation.

In the case of subarachnoid bleeding or bleeding from burst cerebral aneurysms, not only conservative treatment options but neurosurgical interventions as early or delayed operations are used, which are intended to close the source of bleeding from the ruptured aneurysm by the placement of a metal clip. Other treatments may be used.

The therapy may be monitored by electromagnetic imaging and/or ultrasound images without or, optionally, with a contrast agent.

Treatment path B may be used for an ischemic stroke and include the administration of rTPA (recombinant tissue plasminogen activator). The therapy may be monitored by electromagnetic imaging and/or ultrasound images without or, optionally, with a contrast agent. Other treatments may be used.

The conclusion of the treatment for treatment paths A or B may include the acts of: documentation of the diagnosis and therapy in an integrated computer device; transferring the patient to an appropriate location for monitoring; sending the documented diagnosis and therapy data and the data, preferably over a medical data network such as SOARIAN, or DICOM-Modality Performed Procedure Steps (MPPS).

Optionally a control CT may be performed prior to patient discharge.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

What is claimed is:

1. A diagnosis or therapy unit, comprising
at least one of an imaging modality, and two or more devices selected from:
a processor configured to render images from the imaging modality data;
a sensor for detecting patient motion;
a processor configured to correct images for the effects of patient motion;
a patient monitor;
a blood sugar analysis device;
a blood analysis device; and
an image fusion unit; and
a computer and interface for entering patient data,
wherein the imaging modality and two or more of the devices communicate using a data interface.

2. The unit of claim 1, wherein the imaging modality is a C-arch X-ray apparatus.

3. The unit of claim 1, wherein the imaging modality is selected from one or more of a computerized tomography (CT), a magnetic resonance imaging (MRI), a positron emission tomography (PET) scan system, a single photon emission computer tomography (SPECT), or an ultrasonic imaging apparatus.

4. The unit of claim 1, wherein the patient monitor is configured to measure at least one of an electrocardiogram (EKG), a respiration rate, a blood pressure, or a blood oxygen saturation (SpO₂).

5. The unit of claim 1, wherein the image fusion unit is configured to perform at least one of image segmentation, patient motion correction, or image fusion from multiple imaging modalities.

6. The unit of claim 1, further comprising: a data interface with a local area network or a wide area network.

7. The unit of claim 1, comprising three or more devices selected from:
the processor configured to render images from the imaging modality data;
the sensor for detecting patient motion;
the processor configured to correct images for the effects of patient motion;
the patient monitor;
the blood sugar analysis device;
the blood analysis device; and
the image fusion unit.

8. A method of diagnosing a stroke, the method comprising:
positioning the patient on an examination table;
identifying the patient;
attaching sensors from a patient monitor and measuring and recording patient parameters;
obtaining a computerized tomographic image of a portion of the patient using an imaging modality;
reconstructing an image of at least a portion of the patient; and
displaying the image on a monitor or by a projector.

9. The method of claim 8, wherein the imaging modality is a C-arch X-ray apparatus.

10. The method of claim 8, wherein the imaging modality is at least one of a computerized tomography (CT), magnetic resonance imaging (MRI), X-ray, positron emission tomography (PET) scan, single photon emission computer tomography (SPECT), or ultrasound apparatus.

11. The method of claim 8, further comprising correcting imaging data from the imaging modality for the effects of patient motion.

12. The method of claim 8, further including measuring blood values and markers, or retrieving such data previously stored in an accessible memory.

13. The method of claim 8, further comprising segmenting image data obtained from an imaging modality.

14. The method of claim 8, further comprising transmitting data obtained by the sensors over a communications network.

15. The method of claim 14, where the data is transmitted by modulating information on a carrier wave.

16. A method of diagnosing a stroke, the method comprising:
providing a processor configured to:
receive patient identifying information;
measure and record patient parameters obtained from sensors attached to the patient;
receive image data from a first imaging modality employing ultrasound;
receive image data from a second imaging modality employing electromagnetic radiation;
reconstruct an image of at least a portion of the patient from the corrected received image data; and
display the image on a monitor or by a projector.

17. The method of claim 16, wherein the electromagnetic imaging modality is a C-arch X-ray apparatus.

18. The method of claim 16, wherein the processor is configured by instructions stored on a machine readable media.

19. The method of claim 16, further comprising transmitting data received from the sensors over a communications network.

20. The method of claim 16, further comprising receiving information regarding patient motion and correcting the image data for patient motion.

21. The method of claim 19, where the data is transmitted by modulating information on a carrier wave.
